(19) United States
(12) Patent Application Publication
(10) Pub. No.: US 2018/0243007 A1
(43) Pub. Date: Aug. 30, 2018

(54) BONE FIXATOR APPARATUS

(71) Applicant: Clinique Veterinaire La Providence Inc., Saint Jerome (CA)

(72) Inventor: Jymer Max JARAMILLO, Laval (CA)

(21) Appl. No.: 15/442,071

(22) Filed: Feb. 24, 2017

(51) Int. Cl. A61B 17/62; A61B 17/66; A61B 17/64

(52) U.S. Cl. CPC A61B 17/62; A61B 17/645; A61B 17/6458; A61B 17/66

(57) ABSTRACT

A bone fixator apparatus has rings and turnbuckles connected to opposite sections of each ring via opposed right and left handed screws mounted to the rings so that rotation of each turnbuckle modifies a distance between corresponding sections of the rings. On each ring, positioning levers are connected to each screw and mounted on respective sections of the ring. Each positioning lever is movable between an unlocked state allowing the screw to move in relation to the ring and a locked state preventing a movement of the screw. Tensioning levers are mounted on opposite ends of each ring. Each tensioning lever is actionable between an unlocked state allowing mounting one end of a wire to the ring and a locked state causing the end of the wire to be securely attached to the ring. Any tension applied to the wire is maintained by action of the tensioning levers.

### TECHNICAL FIELD

The present disclosure relates to the field of orthopedic apparatuses. More specifically, the present disclosure relates to a bone fixator apparatus having tensioning levers.

### BACKGROUND

The Ilizarov apparatus is a type of external fixation apparatus used in orthopedic surgery. FIG. 1 (Prior Art) is a perspective representation of the Ilizarov apparatus mounted to a broken bone. The Ilizarov apparatus 10 is mounted to a limb (not shown) that includes a bone 12 have a proximal end 14 and a distal end 16. A crack 18 separates the proximal and distal ends 14 and 16 of the bone 12 and the Ilizarov apparatus 10 is mounted to the bone 12 by a practitioner, for example an orthopedic surgeon, a veterinarian, and the like, to maintain the proximal and distal ends 14 and 16 in place while the crack 18 of the bone 10 is healing. The Ilizarov apparatus 10 comprises a pair of rings 20 and 22 mounted around the bone 10 and held together by a number of screws 24 that are substantially parallel to an axis of the bone 10. Each screw 24 is attached at both ends to the rings 20 and 22 by use of nuts 26 on both faces of the rings 20 and 22, on both sides of holes 28 provided in the rings 20 and 22. In order to ensure that the rings 20 and 22 are maintained firmly in place, a pair of nuts 26 attaches each end of each screw 24 on both sides of the rings 20 and 22. The various nuts 26 may be loosened and re-tightened in order to adjust a length such as 30 of each screw 24 between the rings 20 and 22. In this manner, the rings 20 and 22 may be positioned in parallel to one another or at various angles, according to the need of a particular application.

At least one wire such as 32 is mounted to each of the rings 20 and 22 (two wires 32 are mounted to the ring 22 in the example of FIG. 1) so that at least one wire 32 is mounted to the bone 12 on either sides of the crack 18. The practitioner uses a drill (not shown) to piece holes, such as 34, in the proximal and distal ends of the bone 12. The wires 32 are attached to the rings 20 and 22 by clamps 36 having studs 40 and mounted on faces of the rings 20 and 22 by use of opposed nuts 38 placed on opposed faces of the rings 20 and 22. The nuts 38 are screwed to the studs 40, the studs 40 protruding through the rings 20 and 22. The studs 40 have an aperture (not shown) allowing the insertion of ends of the wires 32. The practitioner attaches a first end of the wire 32 to a first clamp 36, locking the first end of the wire 32 by tightening a first nut 38. Then, while applying a tension on a second end of the wire 32, the practitioner tightens a second nut 38 to a second clamp 36. By maintaining both proximal and distal ends 14 and 16 of the bone 12 using the wires 32 under tension, the practitioner can adjust the relative positions of the rings 20 and 22, thereby adjusting the relative positions of the proximal and distal ends 14 and 16 of the bone 12, in order to allow proper healing of the bone 12.

In order to apply a tension a given wire 32₁ mounted to a ring, for example to the ring 20, the practitioner attaches the wire 32₁ at one end to a clamp 36ₐ and tightens the opposed nut (not shown on FIG. 1). The practitioner inserts an opposite end of the wire 32₁ in another opposite clamp 36_{b} and then uses pliers (not shown) or a similar tool to pull on an opposite end of the wire 32₁. An opposed nut 38_{b} is tightened to a stud 40_{b} underneath the clamp 36_{b} while the tension applied on the wire 32₁ is maintained. The practitioner may then stop pulling on the opposite end of the wire 32₁.

Operations required to adjust the distance 30 between the rings 20 and 22, including adjusting and tightening the various nuts 26, and operations required to apply and maintain the tension on the wires 32, including tightening the opposed nuts 38 while pulling on the wires 32, are complex and time intensive, given the context in which the Ilizarov apparatus 10 is normally used, for example in the operating room.

Operation of the Ilizarov apparatus 10 frequently requires the practitioner to be assisted by a trained assistant, in near perfect coordination. In some contexts, for example in a small clinic (developing country, veterinarian clinic), a trained assistant may not always be available.

Therefore, there is a need for improvements in the field of orthopedic apparatuses that compensate for problems related to the complexity of use of the Ilizarov apparatus.

### SUMMARY

According to the present disclosure, there is provided a bone fixator apparatus. The apparatus comprises a pair of rings assembled so to form a regular or irregular cylinder. A wire attachment and a tensioning lever are mounted on each ring. The wire attachment is mounted at one end of the ring and configured to allow securing a first end of a wire to the ring. The tensioning lever is mounted on the ring at an opposite end from the wire attachment. The tensioning lever is actionable between an unlocked state allowing mounting a second end of the wire to the ring and a locked state causing the second end of the wire to be securely attached to the ring. Securing the first end of the wire to the ring and placing the tensioning lever in its locked state allows maintaining any tension applied to the wire.

According to the present disclosure, there is also provided a bone fixator apparatus comprising a pair of rings and a pair of turnbuckles connected to opposite sections of each ring via opposed right and left handed screws mounted to the rings through openings provided in the rings. The screws and the turnbuckles are mounted to the rings so that rotation of each turnbuckle modifies a distance between corresponding sections of the rings.

According to the present disclosure, there is further provided a bone fixator apparatus comprising a pair of rings and a pair of turnbuckles connected to opposite sections of each ring via opposed right and left handed screws mounted to the rings through openings provided in the rings. The screws and the turnbuckles are mounted to the rings so that rotation of each turnbuckle modifies a distance between corresponding sections of the rings. On each ring, a pair of positioning levers is connected to one of the screws and mounted on respective sections of the ring. Each positioning lever is movable between an unlocked state allowing the screw to move in relation to the ring and a locked state preventing a movement of the screw. A pair of tensioning levers is mounted on each ring at opposite ends thereof. Each tensioning lever is actionable between an unlocked state allowing mounting one end of the wire to the ring and a locked state causing the respective end of the wire to be securely attached to the ring. Placing both tensioning levers in their locked states allows maintaining any tension applied to the wire.

The foregoing and other features will become more apparent upon reading of the following non-restrictive description of illustrative embodiments thereof, given by way of example only with reference to the accompanying drawings.

### BRIEF DESCRIPTION OF THE DRAWINGS

Embodiments of the disclosure will be described by way of example only with reference to the accompanying drawings, in which:

FIG. 1 (Prior Art) is a perspective representation of the Ilizarov apparatus mounted to a broken bone;

FIG. 2 is a perspective view of a bone fixator apparatus according to an embodiment;

FIG. 3 is another perspective view of the bone fixator apparatus of FIG. 2;

FIG. 4 is a detailed view of a tensioning lever and of a positioning lever that are parts of the bone fixator apparatus of FIG. 2;

FIG. 5 is a close up view of concave and convex flanges of the positioning lever;

FIG. 6 is a detailed view of a turnbuckle;

FIG. 7 shows detached components for connecting the turnbuckle to the positioning lever;

FIG. 8 is a close up view of a lever; and

FIG. 9 is a view of the bone fixator apparatus in use in an operating room.

Like numerals represent like features on the various drawings.

### DETAILED DESCRIPTION

Various aspects of the present disclosure generally address one or more of the problems related to the complexity of use of the Ilizarov apparatus.

The disclosed bone fixator apparatus may be installed on a limb of a patient and used, for example during surgery, to maintain bones in a desired position. The patient being under anesthesia, a surgeon, veterinarian, or other practitioner places rings of the bone fixator around the limb, connects the rings using turnbuckles, and adjusts a distance and an angle between the rings by rotating the turnbuckles and by adjusting an angle between screws connecting the rings and the turnbuckles. Once the practitioner is satisfied with the placement of the rings, the practitioner uses positioning levers to lock the rings in position. The practitioner then uses a drill to pierce the bone at a level of each ring. Wires are passed through the bone and mounted to the corresponding rings using tensioning levers mounted on the rings. Usually, one end of a wire is fixedly mounted to a corresponding ring using a first tensioning lever. The other end of the wire is placed in position at an opposite end of the ring. The practitioner uses pliers or a similar tool to apply a tension on the wire. While the tension is applied, the practitioner actuates a second tensioning lever to lock the wire in position, the tension being thus maintained on the wire. At any time, the practitioner may release and reapply any one of the positioning levers to better position the rings and the bone in a desired position.

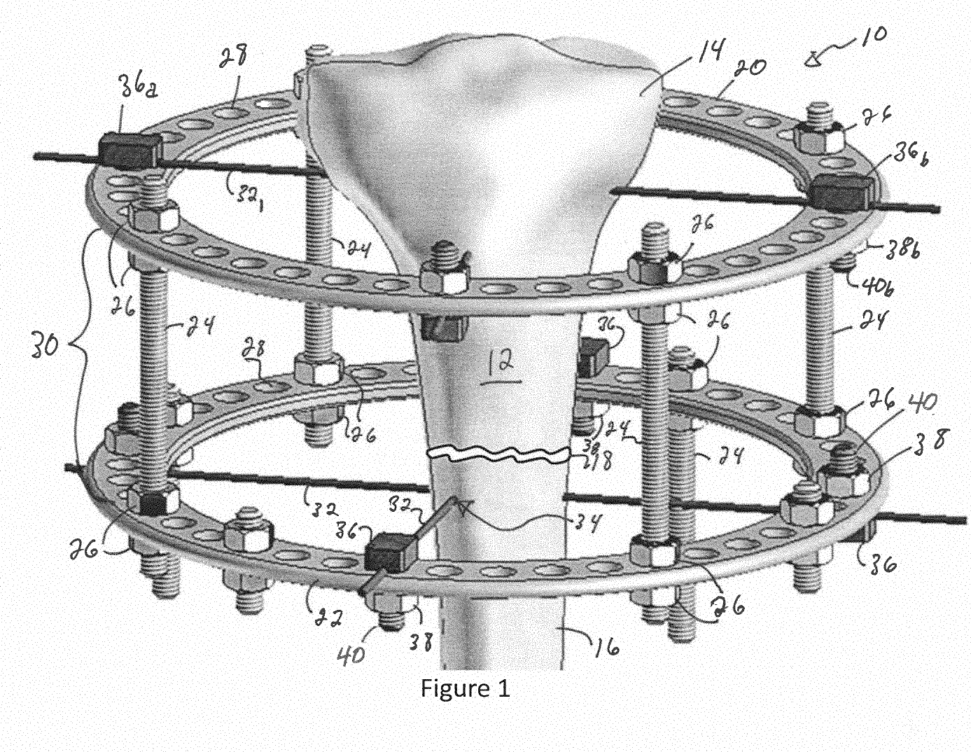

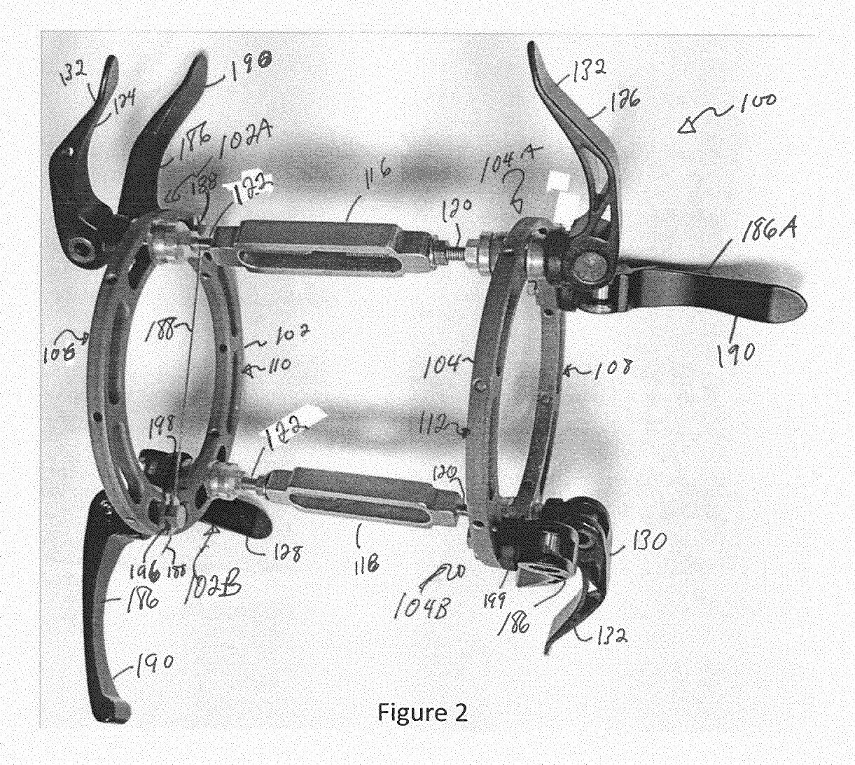

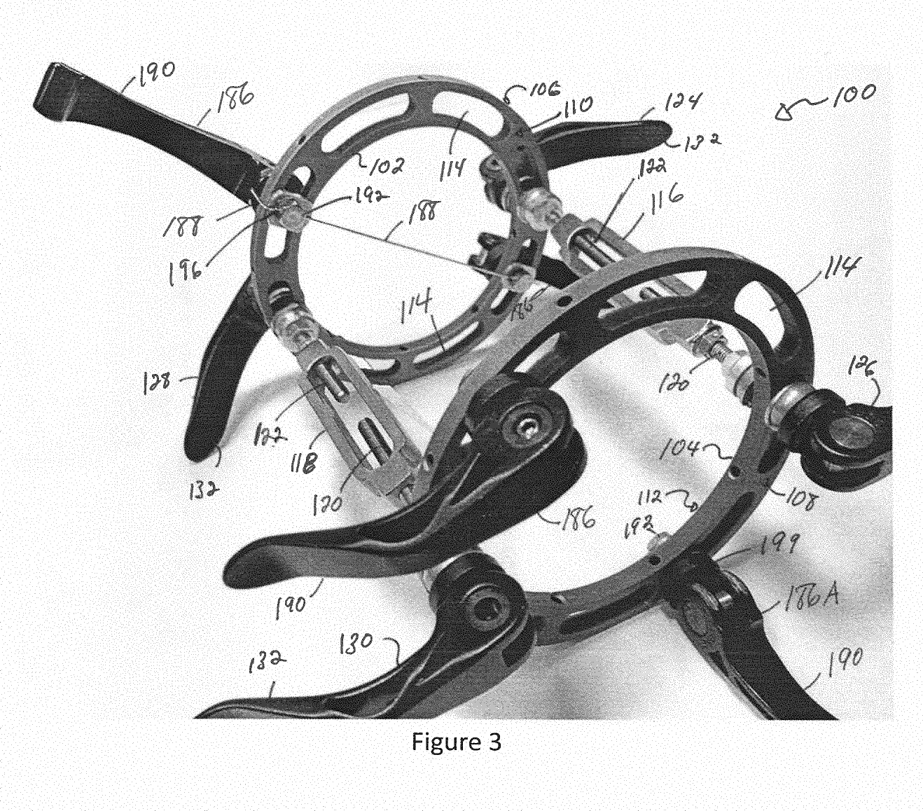

Referring now to the drawings, FIG. 2 is a perspective view of a bone fixator apparatus according to an embodiment. FIG. 3 is another perspective view of the bone fixator apparatus of FIG. 2. An implementation of the bone fixator apparatus will now be described with reference to both FIGS. 2 and 3. A bone fixator apparatus 100 comprises two (2) rings 102 and 104. Each ring 102 and 104 has an outer face 106 or 108 and an inner face 110 or 112. The rings 102 and 104 have openings such as 114 that extend over partial arcs of the rings 102 and 104. Use of circular openings (similar to 28 in FIG. 1) is also contemplated. The rings 102 and 104 are mounted so to form opposite ends of a cylinder. The rings 102 and 104 are not necessarily placed in parallel so they may form an irregular cylinder. Though the rings 102 and 104 as shown are both circular and equally sized, use of unequal size rings and use of non-circular rings is also contemplated.

The rings 102 and 104 may be assembled using screws and nuts, such as the screws 24 and the nuts 26 of FIG. 1. On FIGS. 2 and 3, the rings 102 and 104 are connected by a pair of turnbuckles 116 and 118 via opposed right handed screws 120 and left handed screws 122 mounted to the rings 102 and 104 through the openings 114. The turnbuckle 116 is mounted via openings 114, generally to sections 102A and 104A of the rings 102 and 104. The turnbuckle 118 is mounted via opposite openings 114, generally to opposite sections 102B and 104B of the rings 102 and 104. The screws 120 and 122 and the turnbuckles 116 and 118 are mounted to the rings 102 and 104 so that rotation of the turnbuckle 116 modifies a distance between the sections 102A and 104A while rotation of the turnbuckle 118 modifies a distance between the sections 102B and 104B. Before tightening of the right and left handed screws 120 and 122 to the rings 102 and 104, the screws 120 and 122 may slide within the openings 114 as desired by the user, to modify angles between the turnbuckles 116 and 118 and the rings 102 and 104.

Each one of the opposed right and left screws 120 and 122 of each one of the turnbuckles 116 and 118 is operatively connected to a corresponding positioning lever 124, 126, 128 and 130. The positioning lever 124 is mounted on the section 102A of the ring 102, the positioning lever 126 is mounted on the section 104A of the ring 104, the positioning lever 128 is mounted on the section 1028 of the ring 102, and the positioning lever 130 is mounted on the section 104B of the ring 104. Each positioning lever 124, 126, 128 and 130 is movable between an unlocked state allowing the corresponding screw 120 or 122 to move in relation to the corresponding ring 102 or 104, and a locked state preventing a movement of the corresponding screw 120 or 122 in relation to the corresponding ring 102 or 104. When a given positioning lever 124, 126, 128 or 130 is in the unlocked state, the corresponding screw 120 or 122 is free to translate along the partial arc of a corresponding opening 114 and an angle between the corresponding screw 120 or 122 and the corresponding ring 102 or 104 can be adjusted by the practitioner. Each positioning lever 124, 126, 128 and 130 has a handle 132 that is substantially perpendicular a plane formed by to the ring 102 or 104 when in the unlocked state, and substantially parallel to the plane formed by the ring 102 or 104 when in the locked state.

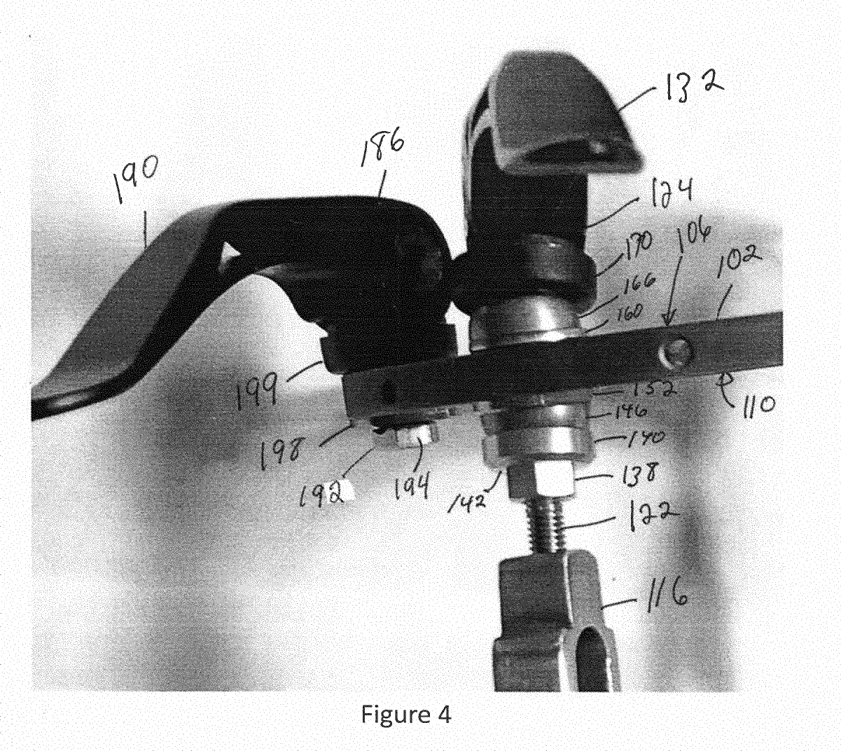

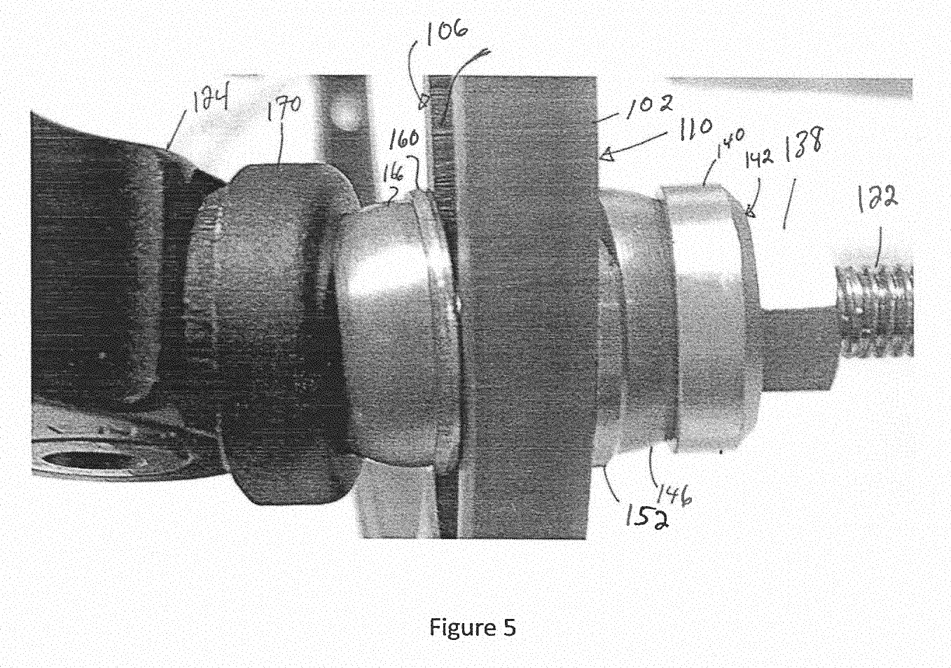

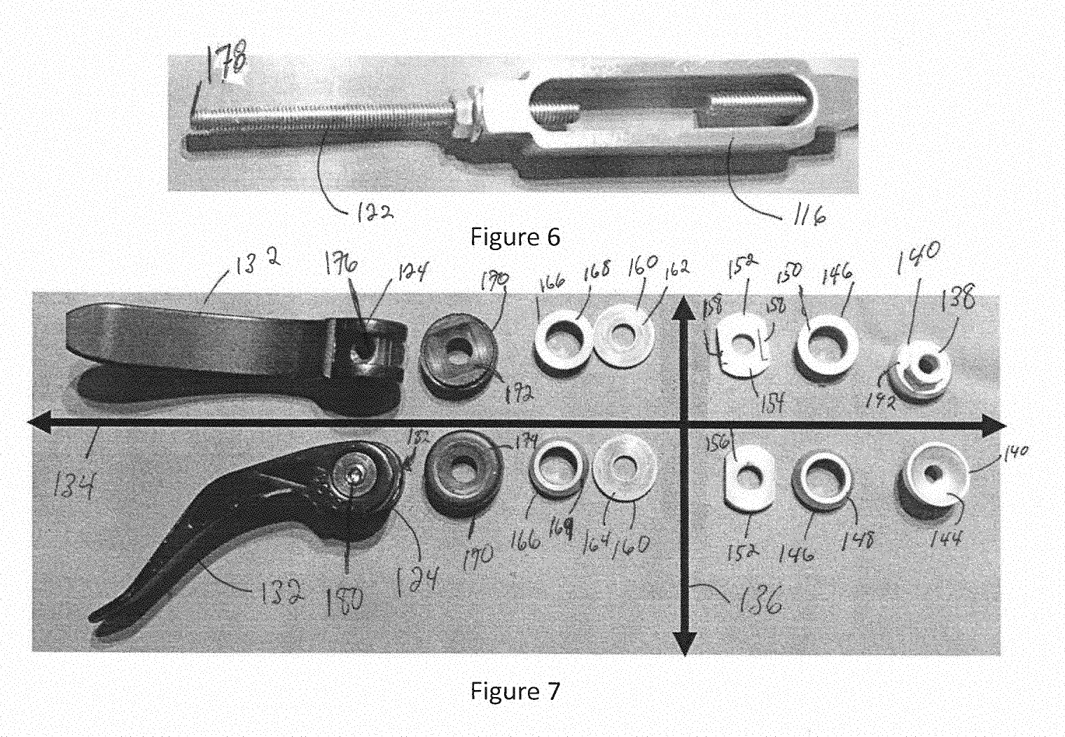

FIG. 4 is a detailed view of a tensioning lever and of a positioning lever that are parts of the bone fixator apparatus of FIG. 2. FIG. 5 is a close up view of concave and convex flanges of the positioning lever. FIG. 6 is a detailed view of a turnbuckle. FIG. 7 shows detached components for connecting the turnbuckle to the positioning lever. Details of the manner in which each turnbuckle 116 or 118 is mounted to the corresponding ring 102 or 104 and to the corresponding positioning lever 124, 126, 128 or 130 will now be described in relation to FIGS. 4 to 7. For illustration purposes, FIGS. 4 to 7 provide partial views of the ring 102 and further show the positioning lever 124 with its handle 132, the turnbuckle 116 and one of the left handed screws 122. In particular, FIG. 7 provides two (2) views of the same positioning lever 124 and of other components (described hereinbelow), above and below a horizontal arrow 134. A vertical arrow 136 separates, on a left side thereof, those components that are mounted on the outer face 106 of the ring 102 and, on a right side thereof, other components that are mounted on the inner face 110 of the ring 102.

The screw 122 at one end of the turnbuckle 116 is mounted to the positioning lever 124 as follows:
A nut 138 is mounted on the screw 122.
A first circular flange 140 is mounted to the screw 122. The first circular flange 140 has a flat face 142 in contact with the nut 138 and a concave face 144 opposite from the flat face 144. Although the nut 138 and the first circular flange 140 may be distinct components, in a variant as shown, the nut 138 and the first circular flange 140 are integrated as a single component.
A second circular flange 146 is mounted on the screw 122. The second circular flange 146 has a convex face 148 in contact with the concave face 144 of the first circular flange 140 and a flat face 150 opposite from the concave face 148.
A first perforated plate 152 having a first flat face 156 in contact with the flat face 150 of the second circular flange 146 and an opposite second flat face 154 in contact with the inner face 110 of the ring 102. In the variant as shown, the first perforated plate 152 has ridges 158 on its second flat face 154. The ridges 158 the ridges are configured to prevent a rotation of the first perforated plate 152 when in contact with the ring 102.
The screw 122 passes through one of the openings 144 of the ring 102.
A second perforated plate 160 is mounted on the screw 122. The second perforated plate 160 has a first flat face 162 in contact with the outer face 106 of the ring 102 and an opposite second flat face 164.
A third circular flange 166 is mounted on the screw 122. The third circular flange 166 has a flat face 168 in contact with the second flat face 164 of the second perforated plate 160 and a convex face 169 opposite from the flat face 168.
A fourth circular flange 170 is mounted on the screw 122. The fourth circular flange 170 a first concave face 174 in contact with the convex face 168 of the third circular flange 166 and an opposite second concave face 172.
The positioning lever 124 has an aperture 176 adapted for the insertion of a tip 178 of the screw 122. The tip 178 of the screw 122 reaches a pivot 180 within the positioning lever 124. The pivot 180 has internal threads (not shown) to receive the tip 178 of the screw 122. A rounded end 182 of the positioning lever 124 makes contact with the second concave face 172 of the fourth circular flange. 170.

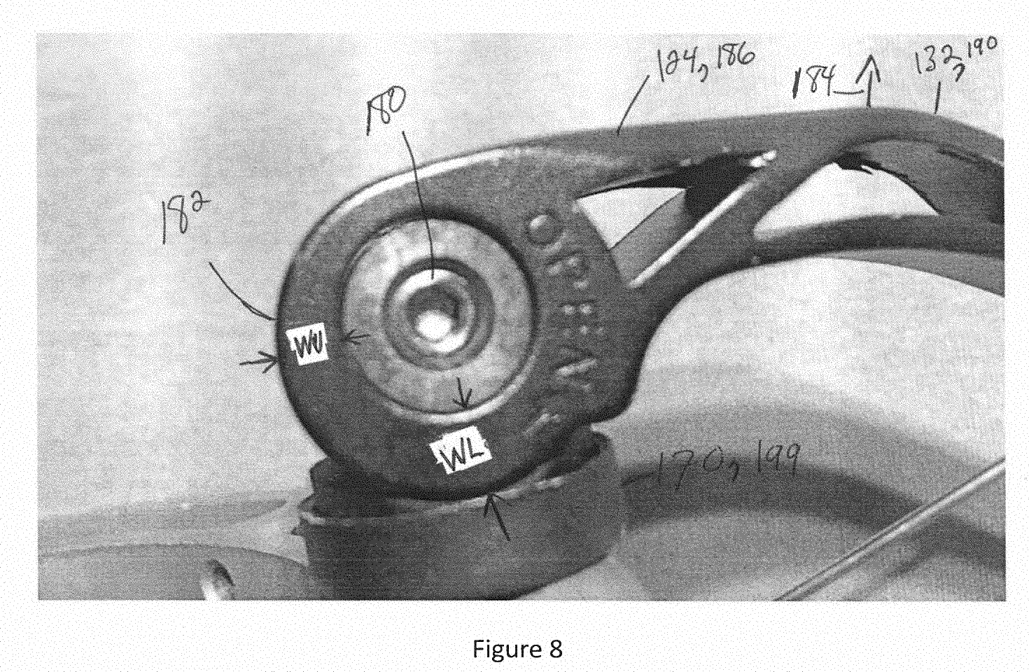

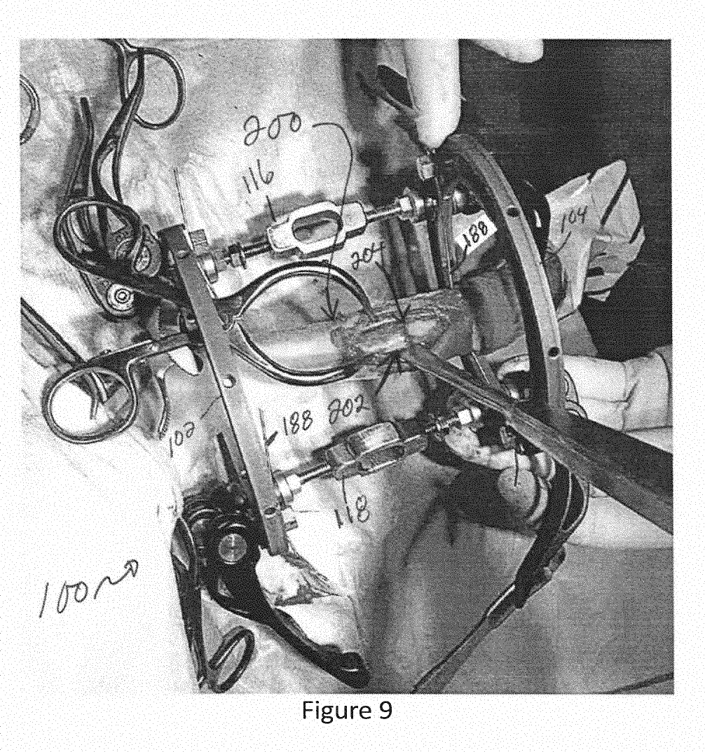

FIG. 8 is a close up view of a lever. In the embodiment as shown and without limitation, the tensioning levers and the positioning levers are constructed in the same manner. The rounded end 182 of the positioning lever 124 forms an eccentric cam, a width ‘WL’ of the rounded end 182 being thicker than a width ‘WU’ of the rounded end 182. When the positioning lever 124 is in the locked state, as shown on FIG. 8, the rounded end 182, due to the width ‘WL’, tends to push on the fourth circular flange 170, causing the positioning lever 124 to apply traction on the screw 122.

Pulling on the handle 132 in the direction of the arrow 184 forces the rounded end 182 of the positioning lever 124 to rotate on the fourth circular flange 170 until the rounded end 182 is in contact with the fourth circular flange 170 at a level of the thinner width ‘WU’. Less pressure is applied by the rounded end 182 on the fourth circular flange 170 and the positioning lever 124 releases the traction on the screw 122. The positioning lever 124 is now in the unlocked state and the screw 122 can slide along the partial arc of the opening 114. The concave and convex shapes of the various flanges 140, 146, 166 and 170 are configured to facilitate a variation of an angle between the screw 122 and the ring 102.

A variant in which the screws 120 and 122 are connected to the rings 102 and 104 using nuts (similar to nuts 26 of FIG. 1, with right or left handed threads for matching the screws 120 and 122), the nuts being mounted on the faces 106, 108, 110 and 112, is also contemplated.

Returning to FIGS. 2 and 3, two (2) tensioning levers 186 are mounted on each of the rings 102 and 104, substantially at opposite ends of the rings 102 and 104. Each tensioning lever 186 forms a wire attachment configured to allow securing one end of a wire 188 to the ring 102 or 104. The tensioning levers 186 are each actionable between an unlocked state allowing mounting one end of the wire 188 to the ring 102 or 104 and a locked state causing the end of the wire 188 to be securely attached to the ring 102 or 104. The tensioning levers 186 are shown in the locked state on FIGS. 2 and 3, their handles 190 being substantially parallel to planes formed by the rings 102 and 104, with the exception of tensioning lever 186A which is shown in its unlocked state, its handle 190 being substantially perpendicular to the plane formed by the ring 104. A first end of the wire 188 is first secured to the ring 102 or 104 by locking a first tensioning lever 186. A second end of the wire 188 is mounted to a second tensioning lever 186 on the same ring 102 or 104. While a tension is applied to the wire 188, for example by use of pliers (not shown), the second tensioning lever 186 is placed in its locked state, allowing any tension applied to the wire to be maintained.

In more details, each tensioning lever 186 is mounted on one face of the corresponding ring 102 or 104, in particular but not exclusively on the outer faces 106 and 108 or the rings 102 and 104. Each tensioning lever 186 is connected to a stubshaft 192 having a head 194 mounted to the inner face 110 or 112 of the ring 102 or 104, and a stud (not shown) extending from the head 194 and protruding through an opening 114 of the ring 102 or 104. The stud of the stubshaft 192 has an aperture 196 dimensioned for receiving an end of the wire 188, the tensioning lever 186 applying a traction on the stud of the stubshaft 192 when in the locked state so to cause the end of the wire 188 to be maintained by compression in a fixed position.

In the embodiment as illustrated, a third perforated plate 198 is mounted on the stud of the stubshaft 192 between the head 194 of the stubshaft 192 and the inner face 110 or 112 of the ring 102 or 104. The wire 188 is maintained by compression between the head 194 of the stubshaft 192 and the third perforated plate 198.

Returning to FIG. 8, the tensioning levers 186 operate in similar fashion to the positioning levers 124, 126, 128 and 130. Each tensioning lever 186 has a pivot 180 and a rounded end 182 forming an eccentric cam, a width ‘WL’ of the rounded end 182 being thicker than a width ‘WU’ of the rounded end 182. The rounded 182 of the tensioning levers 186 each rotate on a concave face of a fifth circular flange 199 mounted on the stud of the stubshaft 192 and in contact with the outer face 106 or 108 of the ring 102 or 104. The stud of the stubshaft 192 has external threads (not shown) received in internal threads of the pivot 180.

When the tensioning lever 186 is in the locked state, as shown on FIG. 8, the rounded end 182, due to the width ‘WL’, tends to push on the fifth circular flange 199, causing the length tensioning lever 186 to apply traction on the stud of the stubshaft 192. At that time, the wire 188 is compressed between the head 194 of the stubshaft 192 and the third perforated plate 198. Pulling on the handle 190 in the direction of the arrow 184 forces the rounded end 182 of the tensioning lever 186 to rotate on fifth circular flange 199 until the rounded end 182 is in contact with the fifth circular flange 199 at a level of the thinner width ‘WU’. Less pressure is applied by the rounded end 182 on the fifth circular flange 199 and the tensioning lever 186 releases the traction on the stud of the stubshaft 192. The tensioning lever 186 is now in the unlocked state and the wire 188 can move in and out of the aperture 196.

In a variant, a single tensioning lever 186 may be installed on each ring 102 and 104. One of the tensioning levers 186 on each ring 102 and 104 may be replaced by a simpler wire attachment such as, for instance, the clamp 36 illustrated on FIG. 1. The practitioner may install a first end of the wire 188 on the ring 102 or 104 using the clamp 36, there being no tension on the wire 188 at the time. The practitioner will then use a tensioning lever 186 at an opposite end of the same ring 102 or 104 to lock the wire 188 while a tension is applied to the wire. Other manners of attaching a first end of the wire 188 to the ring 102 or 104 before locking a second end the wire 188 under tension by use of the tensioning lever 186 are also contemplated. For instance, a wire 188 having at its end a loop (not shown) may be attached to a hook (not shown) before locking an opposite end of the wire 188 under tension by use of the tensioning lever 186.

FIG. 9 is a view of the bone fixator apparatus in use in an operating room. The image of FIG. 9 originates from a veterinary clinic office. The bone fixator apparatus 100 is mounted on a dog's leg 200. A bone 202 has a fracture 204 that is being reduced by a veterinarian. Having installed the wires 188 on the bone 202, the veterinarian operates the turnbuckles 116 and 118 to extend opposite ends of the bone 202 and then to bring them back in an aligned position, as shown on FIG. 9.

Although the bone fixator apparatus as illustrated on FIGS. 2 to 9 comprises two (2) rings connected by two (2) turnbuckles, the present disclosure is not so limited. Implementations of the bone fixator apparatus including three (3) or more rings, each pair of such rings being connected by ordinary screws and nuts or by any number of turnbuckles or by a combination of screws and turnbuckles, are also contemplated. Additional tensioning levers may be placed on a given when it is desired to mount additional wires on the given ring. Combination of the disclosed bone fixator apparatus with additional orthopedic devices conventionally used with the traditional Ilizarov apparatus is also contemplated. Moreover, although the described bone fixator apparatus includes positioning levers and tensioning levers, a simple embodiment may only comprise positioning levers while another simple embodiment may only comprise tensioning levers.

Those of ordinary skill in the art will realize that the description of the bone fixator apparatus is illustrative only and is not intended to be in any way limiting. Other embodiments will readily suggest themselves to such persons with ordinary skill in the art having the benefit of the present disclosure. Furthermore, the disclosed bone fixator apparatus may be customized to offer valuable solutions to existing needs and problems related to the complexity of use of the Ilizarov apparatus.

In the interest of clarity, not all of the routine features of the implementation of the bone fixator apparatus are shown and described. In particular, combinations of features are not limited to those presented in the foregoing description as combinations of elements listed in the appended claims form an integral part of the present disclosure. It will, of course, be appreciated that in the development of any such actual implementation of the bone fixator apparatus, numerous implementation-specific decisions may need to be made in order to achieve the developer's specific goals, such as compliance with application-, system-, and business-related constraints, and that these specific goals will vary from one implementation to another and from one developer to another. Moreover, it will be appreciated that a development effort might be complex and time-consuming, but would nevertheless be a routine undertaking of engineering for those of ordinary skill in the field of orthopedic apparatuses having the benefit of the present disclosure.

The present disclosure has been described in the foregoing specification by means of non-restrictive illustrative embodiments provided as examples. These illustrative embodiments may be modified at will. The scope of the claims should not be limited by the embodiments set forth in the examples, but should be given the broadest interpretation consistent with the description as a whole.

What is claimed is:

1. A bone fixator apparatus, comprising:
a pair of rings assembled so to form a regular or irregular cylinder; and
on each ring:
a wire attachment mounted on the ring, the wire attachment being configured to allow securing a first end of a wire to the ring; and
a tensioning lever mounted on the ring at an opposite end thereof from the wire attachment, the tensioning lever being actionable between an unlocked state allowing mounting a second end of the wire to the ring and a locked state causing the second end of the wire to be securely attached to the ring, wherein securing the first end of the wire to the ring and placing the tensioning lever in its locked state allows maintaining any tension applied to the wire.

2. The apparatus of claim 1, wherein, on each ring, the wire attachment comprises a clamp adapted to be mounted a first face of the ring and having a stud dimensioned to protrude through an opening of the ring, the stud having an aperture dimensioned to allow insertion of the first end of the wire, the wire attachment further comprising a nut mountable to the stud of the clamp on a second face of the ring, wherein tightening of the nut causes the first end of the wire to be maintained by compression in a fixed position.

3. The apparatus of claim 1, wherein the wire attachment comprises another tensioning lever.

4. The apparatus of claim 1, wherein, on each ring, the tensioning lever is mounted a first face of the ring and is connected to a stubshaft having a head mounted to a second face of the ring and a stud extending from the head and protruding through an opening of the ring, the stud of the stubshaft having an aperture dimensioned for receiving the second end of the wire, the tensioning lever applying a traction on the stud of the stubshaft when in the locked state so to cause the second end of the wire to be maintained by compression in a fixed position.

5. The apparatus of claim 4, further comprising a perforated plate mounted on the stud of the stubshaft between the head of the stubshaft and the second face of the ring, the wire being maintained by compression between the head of the stubshaft and the perforated plate.

6. The apparatus of claim 4, wherein an end of the tensioning lever forms an eccentric cam rotating about a pivot, the cam applying pressure on the first face of the ring when in the locked state.

7. The apparatus of claim 6, further comprising a flange mounted on the stud of the stubshaft, between the first face of the ring and the eccentric cam, the eccentric cam being configured to rotate on the flange when moving between the unlocked and locked states.

8. The apparatus of claim 6, wherein the stud of the stubshaft is connected to the pivot.

9. The apparatus of claim 1, further comprising a pair of turnbuckles connected to opposite sections of each ring via opposed right and left handed screws mounted to the rings through openings provided in the rings, the screws and the turnbuckles being mounted to the rings so that rotation of each turnbuckle modifies a distance between corresponding sections of the rings.

10. The apparatus of claim 9, wherein the openings provided in the rings extend over partial arcs of the rings.

11. The apparatus of claim 9, wherein each one of the opposed right and left screws of each one of the turnbuckles is attached to a corresponding section of a corresponding ring using a pair of nuts mounted to the screw on opposite faces of the corresponding ring.

12. The apparatus of claim 9, wherein each one of the opposed right and left screws of each one of the turnbuckles is operatively connected to a corresponding positioning lever mounted on a corresponding section of a corresponding ring, each positioning lever being movable between an unlocked state allowing the screw to move in relation to the corresponding ring and a locked state preventing a movement of the screw in relation to the corresponding ring.

13. The apparatus of claim 12, wherein the positioning levers, when in the unlocked states, allow adjusting angles between the corresponding screws and the corresponding rings.

14. The apparatus of claim 12, wherein each screw connecting each end of each turnbuckle is mounted to the corresponding positioning lever using:
a nut mounted on the screw;
a first circular flange mounted to the screw, the first circular flange having a flat face in contact with the nut and a concave face opposite from the flat face;
a second circular flange mounted on the screw, the second circular flange having a convex face in contact with the concave face of the first circular flange and a flat face opposite from the concave face;
a first perforated plate having a first flat face in contact with the flat face of the second circular flange and an opposite second flat face in contact with an inner face of the corresponding ring;
a second perforated plate mounted on the screw, the second perforated plate having a first flat face in contact with an outer face of the corresponding ring and an opposite second flat face;
a third circular flange mounted on the screw, the third circular flange having a flat face in contact with the second flat face of the second perforated plate and a convex face opposite from the flat face; and
a fourth circular flange mounted on the screw, the fourth circular flange having a first concave face in contact with the convex face of the third circular flange and an opposite second concave face;
wherein the corresponding positioning lever is mounted to an end of the screw and in contact with the second concave face of the fourth circular flange, the positioning lever being movable between an unlocked state and a locked state, the positioning lever applying traction on the screw when in the locked state.

15. The apparatus of claim 14, wherein the nut mounted on the screw and the first circular flange are integrated as a single component.

16. The apparatus of claim 14, wherein the first perforated plate has ridges on the second flat face in contact with the inner face of the corresponding ring, the ridges being configured to prevent a rotation of the first perforated plate when in contact with the corresponding ring.

17. The apparatus of claim 12, wherein an end of each positioning lever forms an eccentric cam rotating about a pivot, the cam applying pressure on the corresponding ring and applying traction on an end of a corresponding screw when in the locked state.

18. A bone fixator apparatus, comprising:
a pair of rings; and
a pair of turnbuckles connected to opposite sections of each ring via opposed right and left handed screws mounted to the rings through openings provided in the rings, the screws and the turnbuckles being mounted to the rings so that rotation of each turnbuckle modifies a distance between corresponding sections of the rings.

19. A bone fixator apparatus, comprising:
a pair of rings;
a pair of turnbuckles connected to opposite sections of each ring via opposed right and left handed screws mounted to the rings through openings provided in the rings, the screws and the turnbuckles being mounted to the rings so that rotation of each turnbuckle modifies a distance between corresponding sections of the rings; and
on each ring:
a pair of positioning levers operatively connected to a respective one of the screws and mounted on respective sections of the ring, each positioning lever being movable between an unlocked state allowing the respective one of the screws to move in relation to the ring and a locked state preventing a movement of the respective one of the screws in relation to the ring; and
a pair of tensioning levers mounted on the ring at opposite ends thereof, each tensioning lever being actionable between an unlocked state allowing mounting a respective end of a wire to the ring and a locked state causing the respective end of the wire to be securely attached to the ring, wherein placing both tensioning levers in their locked states allows maintaining any tension applied to the wire.
